(19) United States
(12) Patent Application Publication
(10) Pub. No.: US 2009/0140239 A1
(43) Pub. Date: Jun. 4, 2009

(54) COMPOUNDS FOR ORGANIC SEMICONDUCTOR DEVICE HAVING TRIAZINE GROUP, ORGANIC SEMICONDUCTOR THIN FILM AND ORGANIC SEMICONDUCTOR DEVICE COMPRISING THE SAME, AND METHODS OF PREPARING THEM

(75) Inventors: Hyo Young Lee, Daejeon-city (KR); Do Hyun Kim, Daejeon-city (KR); Hyun Jung Do, Daejeon-city (KR)

Correspondence Address: BLAKELY SOKOLOFF TAYLOR & ZAFMAN LLP, 1279 OAKMEAD PARKWAY, SUNNYVALE, CA, 94085-4040, US

(73) Assignee: Electronics and Telecommunications Research Institute

(21) Appl. No.: 12/275,030

(22) Filed: Nov. 20, 2008

Related U.S. Application Data
(62) Division of application No. 11/085,886, filed on Mar. 21, 2005, now abandoned.

(30) Foreign Application Priority Data
Oct. 11, 2004 (KR) 10-2004-0081117

(51) Int. Cl. H01L 51/00; C07D 251/24

(52) U.S. Cl. 257/40; 544/216; 257/E51.001

(57) ABSTRACT

A compound for organic semiconductor devices having a triazine group, an organic semiconductor thin film and an organic semiconductor device comprising the same, and methods of preparing them are provided. The compound for organic semiconductor devices is represented by the following Formula:

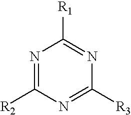

where each of R₁, R₂ and R₃ is a perfluorophenylene derivative.

This application claims priority from Korean Patent Application No. 10-2004-0081117, filed on Oct. 11, 2004, in the Korean Intellectual Property Office, the disclosure of which is incorporated herein in its entirety by reference.

### BACKGROUND OF THE INVENTION

1. Field of the Invention

The present invention relates to an organic semiconductor material and a method of preparing the same, and an organic semiconductor device comprising the same, and more particularly, to a compound for n-type organic semiconductor devices substituted by fluorine atoms, an organic semiconductor thin film and organic semiconductor device comprising the same, and methods of preparing them.

2. Description of the Related Art

After it was reported that an organic compound having a conjugation of π-electrons exhibits semiconductor characteristics, research on an organic semiconductor material has been carried out. In particular, an organic electroluminescent diode device for a display is being commercialized. Many efforts to develop an organic semiconductor material having various properties are made in order to improve an organic semiconductor technology in new fields such as an organic thin film transistor, an organic solar light and molecular electronics.

The organic semiconductor materials are divided into a p-type semiconductor involved in hole injection and hole transport and an n-type semiconductor involved in electron injection and electron transport. In organic semiconductor devices, an organic electroluminescent device adopts an electron injection layer and an electron transport layer, an organic solar cell adopts an n-type material with p-n junction, and an organic thin film transistor device adopts an n-type semiconductor material such as n-type thin film channel material, Regarding n-type organic semiconductor material, hydrogen bonded to carbon in a molecule easily reacts with oxygen in air to be oxidized due to an electron-attracting property, thereby resulting in a loss of inherent properties of the compound. In particular, as the ability of attracting electrons grows stronger, an oxidation of the organic compound molecule increases. For this reason, a material having strong n-type organic semiconductor characteristics is relatively difficult to be developed in comparison of a p-type.

In order to resolve the above problems, an effort to improve performance of the organic semiconductor devices by substituting hydrogen bonded to carbon by fluorine, which strongly bonds to carbon, to improve thermodynamical stability and result in reducing an oxidation caused with external oxygen and humidity has been made. However, the organic molecules including fluorine is difficult to be synthesized.

It is known that a central molecular group should attract well electrons from the periphery in order to be a good n-type organic semiconductor material. Recently, an n-type semiconductor material having only C—F substituents by carbon-carbon coupling of an aromatic ring group including fluorines on a benzene ring is developed. However, the structure based on the benzene ring does not provide sufficient electron negativity to attract electrons. In conventional technologies, an n-type organic semiconductor material based on a molecular group having sufficiently high electron negativity cannot be developed due to a difficult carbon-carbon coupling of a molecular group having a high electron negativity.

### SUMMARY OF THE INVENTION

The present invention provides a compound for organic semiconductor devices having a new structure based on a molecular group with high electron negativity.

The present invention also provides a method of preparing the compound for organic semiconductor devices having a new structure based on a molecular group with high electron negativity.

The present invention also provides an organic semiconductor thin film composed of the compound for organic semiconductor devices having a new structure.

The present invention also provides a method of forming the organic semiconductor thin film composed of the compound for organic semiconductor devices having a new structure.

The present invention also provides an organic semiconductor having the semiconductor film composed of the compound for organic semiconductor devices having a new structure.

According to an aspect of the present invention, there is provided a compound for organic semiconductor devices represented by Formula (1):

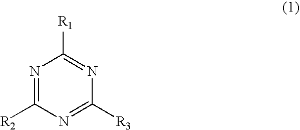

where each of R₁, R₂ and R₃ is a perfluorophenylene derivative.

Each of R₁, R₂ and R₃ may be represented by Formula (2), Formula (3) or Formula (4):

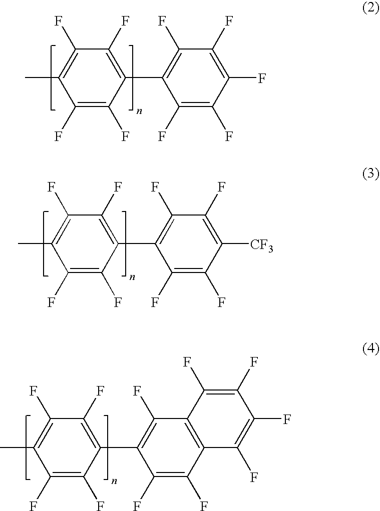

where n is an integer from 0 to 20.

According to another aspect of the present invention, there is provided a method of preparing the compound for organic semiconductor devices represented by Formula (1) above, the method including coupling a 1,3,5-triazine derivative and a perfluorophenylene derivative.

The 1,3,5-triazine derivative may be 2,4,6-trichloro-1,3,5-triazine.

According to another aspect of the present invention, there is provided an organic semiconductor thin film composed of the compound represented by Formula (1) above.

According to another aspect of the present invention, there is provided a method of forming the organic semiconductor thin film, the method including: preparing the compound for organic semiconductor devices represented by Formula (1); and forming a thin film composed of the compound on a substrate. The substrate may be composed of ITO/glass, metal electrode/glass, or metal electrode/silicon. The thin film may be formed by vacuum deposition, spin coating, ink-jet coating or screen printing.

According to another aspect of the present invention, there is provided an organic semiconductor device comprising the semiconductor film composed of the compound represented by Formula (1). The semiconductor film may constitute an electron injection layer or electron transport layer of an organic electroluminescent device. The semiconductor film may constitute a channel layer of an n-type transistor.

The compound according to an embodiment of the present invention has lower energy levels (highest occupied molecular orbital (HOMO), lowest unoccupied molecular orbital (LUMO)) than any conventional n-type organic semiconductor material due to a triazine group having an electron-attracting property. Thus, the compound can be effectively applied to n-type organic semiconductor devices which require the low energy levels. As the length of conjugated substituents of the compound according to an embodiment of the present invention varies, the LUMO and HOMO energy levels can be adjusted. Thus, the compound can be appropriately applied to the n-type organic semiconductor devices according to types and using purposes thereof.

### BRIEF DESCRIPTION OF THE DRAWINGS

The above and other features and advantages of the present invention will become more apparent by describing in detail exemplary embodiments thereof with reference to the attached drawings in which:

FIG. 1A is a reaction scheme for synthesizing a 2,4,6-tris-perfluorophenylene-[1,3,5]triazine derivative according to an embodiment of a method of the present invention;

FIG. 1B is a reaction scheme for synthesizing a 2,4,6-tris-perfluorophenylene-[1,3,5]triazine derivative according to another embodiment of a method of the present invention;

FIG. 1C is a reaction scheme for synthesizing a 2,4,6-tris-perfluorophenylene-[1,3,5]triazine derivative according to still another embodiment of a method of the present invention;

FIG. 2 is an UV spectrum of Compound IIa;

FIG. 3 is a photoluminescence (PL) spectrum of Compound IIa;

FIG. 4 is a fourier transform-infrared (FT-IR) spectrum of Compound IIa;

FIG. 5A is a thermogravimetric analysis (TGA) spectrum of Compound IIa;

FIG. 5B is a differential scanning calorimetry (DSC) spectrum of Compound IIa;

FIG. 6 is a ¹³C-nuclear magnetic resonance (NMR) spectrum of Compound IIa;

FIG. 7 is a ¹⁹F-NMR spectrum of Compound IIa;

FIG. 8 is a mass spectrometry (MS) spectrum of Compound IIa;

FIG. 9 is the results of cyclic voltametry (CV) of Compound IIa; and

FIG. 10 is a cross-sectional view for explaining a method of forming an organic semiconductor thin film according to an embodiment of the present invention.

### DETAILED DESCRIPTION OF THE INVENTION

In the present invention, compounds having a triazine group rather than a benzene group, which is conventionally known, as a central molecular group are provided. In the present invention, a novel 2,4,6-tris-perfluorophenylene-[1,3,5]triazine compound and derivatives thereof are synthesized to provide a material for n-type organic semiconductor devices without difficulty in synthesis according to conventional technology.

The present invention provides a novel 2,4,6-tris-perfluorophenylene-[1,3,5]triazine derivative compounds represented by the following Formula (1) by coupling a fluorine-substituted phenyl group to a 1,3,5-triazine derivative strongly attracting external electrons, for example, a 2,4,6-trichloro-1,3,5-triazine derivative.

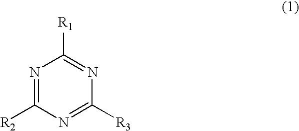

where each of R₁, R₂ and R₃ is a perfluorophenylene derivative. Each of R₁, R₂ and R₃ may be represented by Formula (2), Formula (3) or Formula (4):

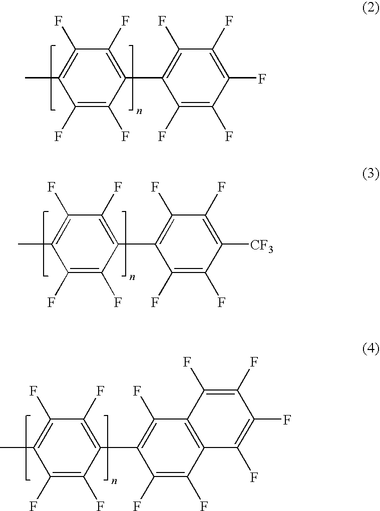

where n is an integer from 0 to 20.

The present invention also provides an organic semiconductor thin film and an organic semiconductor device composed of the compound represented by Formula (1). The organic semiconductor device has at least one organic functional layer interposed between a pair of electrodes, wherein the organic functional layer includes the compound represented by Formula (1).

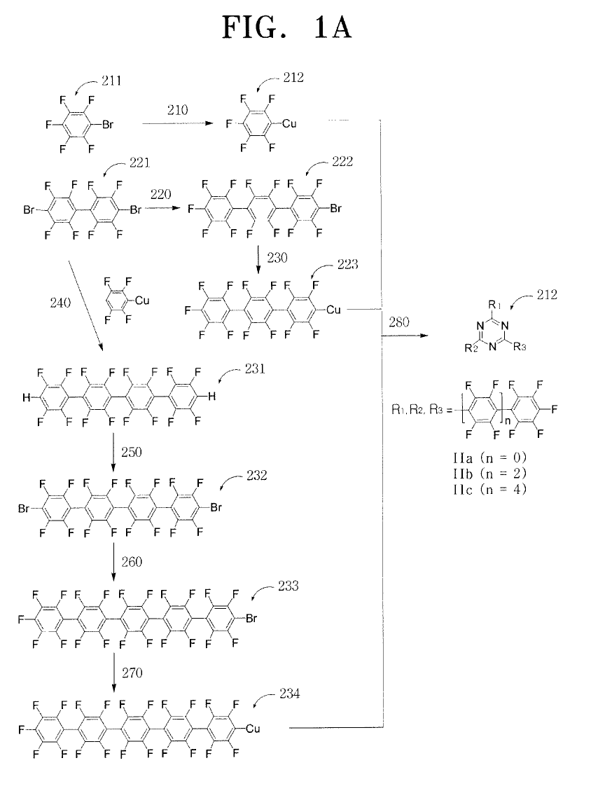

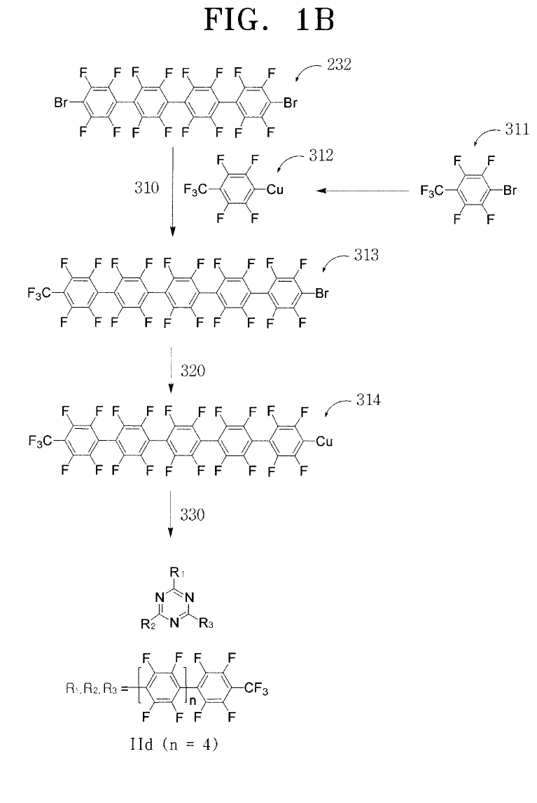

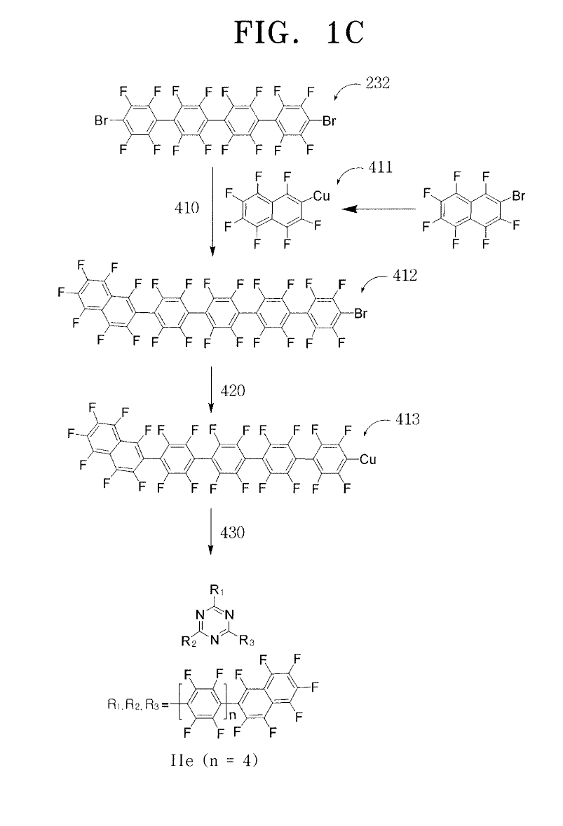

FIG. 1A is a reaction scheme for synthesizing a 2,4,6-tris-perfluorophenylene-[1,3,5]triazine derivative according to an embodiment of the method of the present invention.

A hydrogen-substituted phenyl group is reacted with 2,4,6-trichloro-1,3,5-triazine compound through Suzuki coupling to synthesize the target compound with high yield. However, a reaction of a fluorine-substituted phenyl group, which has a high electron negativity, and 2,4,6-trichloro-1,3,5-triazine compound cannot be performed under the same conditions as in the above reaction. Since the fluorine functional group having inductive effect attracts π-electrons in the phenyl group, coupling reaction of aromatic carbon-carbon is used to be interrupted in comparison of not having electron withdrawing groups. In the present invention, an organometallic compound containing copper is used for the reaction of the fluorine-substituted phenyl group and 2,4,6-trichloro-1,3,5-triazine compound. Bromoperfluorophenylene compounds (Compound 222 and Compound 233 of FIG. 1A) and perfluorophenyleneyl-copper compounds (Compound 212, Compound 223 and compound 234 of FIG. 1A) are prepared according to methods disclosed in a literature (Journal of American Chemical Society, 2000, 122, 10240-10241). The compound represented by Formula (1) can be obtained by a radical reaction of a perfluorophenyleneyl-copper and 2,4,6-trichloro-1,3,5-triazine compound. All novel compounds shown in the reaction scheme of FIG. 1A are identified as desired compounds through hydrogen/carbon nuclear magnetic resonance (NMR) and mass spectrometry (MS) spectrums.

The synthesis procedure shown in FIG. 1A will now be described with reference to specific Synthesis Examples.

### SYNTHESIS EXAMPLE 1

### Procedure 220 of FIG. 1A

Magnesium (7.54 mmol) was put into a 500 mL three-neck round-bottom flask equipped with a reflux condenser and a magnetic stirrer under anhydrous conditions. The reaction vessel was slowly heated to reflux and bromoperfluorophenylene compound (7.54 mmol) (Compound 211 of FIG. 1A) dissolved in anhydrous tetrahydrofuran (THF) (15 mL) was slowly added thereto with a syringe under nitrogen atmosphere. The reaction mixture was stirred at room temperature for about 1 hour and anhydrous Cu(I) Br (15.2 mmol), dioxane (8 mL), and dibromofluorophenylene (11.6 mmol) (Compound 221 of FIG. 1A) dissolved in anhydrous toluene (25 mL) were added thereto. The resulting mixture was heated at 80° C. for 24 hours. It is very important to maintain an inert atmosphere for an oxygen-free atmosphere throughout the reaction. In the present Example, the oxygen-free atmosphere was produced by maintaining a nitrogen atmosphere throughout the reaction. After the reaction was completed, each of the concentrated products was purified by silica gel chromatography with a solvent system of 10% dichloromethane and hexane to obtain bromoperfluorophenylene (Compound 222 of FIG. 1A) (yield: 51%). The obtained compound was identified as a desired product (Compound 222 of FIG. 1A) through MS spectrum. The data was as follows.

Compound 222: m/z (%): 541.90 (M⁺ 100.0%), 543.90 (99.2%), 542.90 (20.0%), 544.90 (19.5%), 545.90 (1.8%).

### SYNTHESIS EXAMPLE 2

### Procedure 210 of FIG. 1A

In the same manner as in Synthesis Example 1, magnesium (6.10 mmol) was put into a 100 mL two-neck round-bottom flask equipped with a reflux condenser and a magnetic stirrer under anhydrous conditions. The reaction vessel was slowly heated to reflux and bromoperfluorophenylene compound (6.10 mmol) (Compound 211 of FIG. 1A) dissolved in anhydrous tetrahydrofuran (THF) (7 mL) was slowly added thereto with a syringe under nitrogen atmosphere. The resulting mixture was stirred at room temperature for about 1 hour to obtain a brown solution. The brown solution was transferred to a 100 mL two-neck round-bottom flask containing anhydrous Cu(I)Br (1.82 g, 12.7 mmol) with a metal tube. The mixture was stirred at room temperature for about 1 hour, and then dioxane (4-10 mL) was added thereto. The resultant was stirred at room temperature for about 1 hour to obtain a pale brown solution. The resulting perfluorophenyleneyl-copper compound (Compound 212 of FIG. 1A) was used in a subsequent reaction without purification due to its sensitivity to air and humidity.

### SYNTHESIS EXAMPLE 3

### Procedure 230 of FIG. 1A

A pale brown solution was obtained in the same manner as in Synthesis Example 2, except that Compound 222 was used instead of Compound 211. The resulting perfluorophenyleneyl-copper compound (compound 223 of FIG. 1A) was used in a subsequent reaction without purification due to its sensitivity to air and humidity.

### SYNTHESIS EXAMPLE 4

### Procedure 260 of FIG. 1A

Bromoperfluorophenylene (Compound 233 of FIG. 1A) (yield: 53%) was obtained in the same manner as in Synthesis Example 1, except that Compound 232 of FIG. 1A was used as dibromofluorophenylene. The obtained compound was identified as a desired product (Compound 233 of FIG. 1A) through MS spectrum. The data was as follows.

Compound 233: m/z (%): 837.88 (M⁺ 100.0%), 839.88 (97.3%), 838.89 (33.4%), 840.89 (33.0%), 839.89 (5.4%), 841.89 (5.2%).

### SYNTHESIS EXAMPLE 5

### Procedure 270 of FIG. 1A

A pale brown solution was obtained in the same manner as in Synthesis Example 2, except that Compound 233 was used instead of Compound 211. The resulting perfluorophenyleneyl-copper compound (Compound 234 of FIG. 1A) was used in a subsequent reaction without purification due to its sensitivity to air and humidity.

### SYNTHESIS EXAMPLE 6

### Procedure 280 of FIG. 1A

2,4,6-Trichloro-1,3,5-triazine (150 mg, 0.68 mmol) dissolved in anhydrous toluene (15-20 mL) was added to the brown solutions obtained in Synthesis Examples 2, 3 and 5, respectively. Each of the reaction mixtures was stirred for about 6 days while maintaining it at about 100° C. After the reaction was completed, the product was filtered with a column chromatography tube containing an activated carbon (200-300 meshes) to remove solids. The filtrate was concentrated. Each of the concentrated products was purified by a silica gel chromatography with a solvent system of 5-20% dichloromethane and hexane. As a result, white solid Compound IIa (250 mg, yield: 53%), Compound IIb (650 mg, yield: 54%) and Compound IIc (900 mg, yield: 47%), which were 2,4,6-tris-perfluorophenylene-[1,3,5]triazine derivatives (Compound 212 of FIG. 1A), were obtained. The products were identified to have the same structure as that of Compound IIa through NMR and MS spectrums. The data were given below. The structures of Compound IIb and Compound IIc were identified through MS spectrum (MALDI-TOF).

Compound IIa: ¹⁹F NMR (CDCl₃) (ppm): −140.5, −147.9, −160.4; ¹³C NMR (CDCl₃) (ppm): 167.2, 147.1, 144.6, 142.2, 139.3, 136.8, 111.6; MS (EI), m/z (%): 124(8), 193(100), 341(7), 579 (M⁺ 40).

Compound IIb: MS (MALDI-TOF), m/z (%): 1466.9 (M⁺ 100.0%), 1468.0 (63.4%), 1469.0 (19.7%), 1470.0 (4.2%), 1467.9 (1.1%).

Compound IIc: MS (MALDI-TOF), m/z (%): 2355.9 (M⁺ 100.0%), 2354.9 (95.6%), 2356.9 (51.7%), 2357.9 (17.6%), 2358.9 (4.5%).

FIG. 1B is a reaction scheme for synthesizing 2,4,6-tris-perfluorophenylene-[1,3,5]triazine derivative according to another embodiment of the method of the present invention.

The synthesis procedure shown in FIG. 1B will now be described with reference to specific Synthesis Examples.

### SYNTHESIS EXAMPLE 7

### Procedure 310 of FIG. 1B

Magnesium (7.54 mmol) was put into a 500 mL three-neck round-bottom flask equipped with a reflux condenser and a magnetic stirrer under anhydrous conditions. The reaction vessel was slowly heated to reflux and 1-bromo-2,3,5,6-tetrafluoro-4-trifluoromethyl-benzene (7.54 mmol) (Compound 311 of FIG. 1B) dissolved in anhydrous THF (15 mL) was slowly added thereto with a syringe under nitrogen atmosphere. The reaction mixture was stirred at room temperature for about 1 hour and anhydrous Cu(I)Br (15.2 mmol), dioxane (8 mL), and dibromofluorophenylene (11.6 mmol) (Compound 232 of FIG. 1B) dissolved in anhydrous toluene (25 mL) were added thereto. The resulting mixture was heated at 80° C. for 24 hours. An oxygen-free atmosphere was produced by maintaining a nitrogen atmosphere throughout the reaction. After the reaction was completed, each of the concentrated products was purified by silica gel chromatography with a solvent system of 10% dichloromethane and hexane. As a result, 4″″-bromo-2,3,5,6,2′,3′,5′,6′,2″,3″,5″,6″,2′″,3′″,5′″,6′″,2″″,3″″,5″″,6″″-eicosafluoro-4-trifluoromethyl-[1,1′;4′,1″;4″,1′″;4′″,1″″]quinquephenyl (Compound 313 of FIG. 1B) (yield: 51%) was obtained. The obtained product was identified as a desired compound (Compound 313 of FIG. 1B) through MS spectrum. The data was as follows.

MS (MALDI) m/e: 889.9 (100.0%), 887.9 (97.1%), 888.9 (33.5%), 890.9 (33.2%), 891.9 (5.5%)

### SYNTHESIS EXAMPLE 8

### Procedure 320 of FIG. 1B

In the same manner as in Synthesis Example 7, magnesium (6.10 mmol) was put into a 100 mL two-neck round-bottom flask equipped with a reflux condenser and a magnetic stirrer under anhydrous conditions. The reaction vessel was slowly heated to reflux and compound 313 of FIG. 1B (6.10 mmol) dissolved in anhydrous THF (7 mL) was slowly added thereto with a syringe under nitrogen atmosphere. The resulting mixture was stirred at room temperature for about 1 hour to obtain a brown solution. The brown solution was transferred to a 100 mL two-neck round-bottom flask containing anhydrous Cu(I)Br (1.82 g, 12.7 mmol) with a metal tube. The mixture was stirred at room temperature for about 1 hour, and then dioxane (4-10 mL) was added thereto. The resultant was stirred at room temperature for about 1 hour to obtain a pale brown solution. The resulting compound (Compound 314 of FIG. 18) was used in a subsequent reaction without purification due to its sensitivity to air and humidity.

### SYNTHESIS EXAMPLE 9

### Procedure 330 of FIG. 1B

2,4,6-Trichloro-1,3,5-triazine (150 mg, 0.68 mmol) dissolved in anhydrous toluene (20 mL) was added to the brown solution (compound 314 of FIG. 1B) prepared in Synthesis Examples 8. The reaction mixture was stirred for about 6 days while maintaining it at about 100° C. After the reaction was completed, the product was filtered with a column chromatography tube containing an activated carbon (200-300 meshes) to remove solids. The filtrate was concentrated. The concentrated product was purified by a silica gel chromatography with a solvent system of 20% dichloromethane and hexane. As a result, white solid Compound IId (1.04 g, yield: 51%), which was a 2,4,6-tris-perfluorophenylene-[1,3,5]triazine derivative, was obtained. The product was identified to have the same structure as that of Compound IId through MS spectrum (MALDI-TOF).

MS (MALDI): m/e 2505.9 (100.0%), 2504.9 (92.7%), 2506.9 (53.4%), 2507.9 (18.8%), 2508.9 (4.9%), 2509.9 (1.0%)

FIG. 1C is a reaction scheme for synthesizing 2,4,6-tris-perfluorophenylene-[1,3,5]triazine derivative according to still another embodiment of the method of the present invention.

The synthesis procedure shown in FIG. 1C will now be described with reference to specific Synthesis Examples.

### SYNTHESIS EXAMPLE 10

### Procedure 410 of FIG. 1C

Magnesium (7.54 mmol) was put into a 500 mL three-neck round-bottom flask equipped with a reflux condenser and a magnetic stirrer under anhydrous conditions. The reaction vessel was slowly heated to reflux and 2-bromo-1,3,4,5,6,7,8-heptafluoro-naphthalene compound (7.54 mmol) (Compound 411 of FIG. 1C) dissolved in anhydrous THF (15 mL) was slowly added thereto with a syringe under nitrogen atmosphere. The reaction mixture was stirred at room temperature for about 1 hour and anhydrous Cu(I)Br (15.2 mmol), dioxane (8 mL), and dibromofluorophenylene (11.6 mmol) (Compound 232 of FIG. 1C) dissolved in anhydrous toluene (25 mL) were added thereto. The resulting mixture was heated at 80° C. for 24 hours. An oxygen-free atmosphere was produced by maintaining a nitrogen atmosphere throughout the reaction. After the reaction was completed, each of the concentrated products was purified by silica gel chromatography with a solvent system of 10% dichloromethane and hexane. As a result, 4″″-bromo-2,3,5,6,2′,3′,5′,6′,2″,3″,5″,6″,2″″,3′″,5″″,6′″-hexadecafluoro-4-(1,3,4,5,6,7,8-heptafluoro-naphthalene-2-yl)-[1,1′;4′,1″;4″,1′″]quaterphenyl (Compound 412 of FIG. 1C) (yield: 47%) was obtained. The obtained product was identified as a desired compound (Compound 412 of FIG. 1C) through MS spectrum. The data was as follows.

MS (MALDI) m/e: 925.9 (100.0%), 923.9 (96.0%), 924.9 (36.3%), 926.9 (36.1%), 927.9 (6.5%)

### SYNTHESIS EXAMPLE 11

### Procedure 420 of FIG. 1C

In the same manner as in Synthesis Example 10, magnesium (6.10 mmol) was put into a 100 mL two-neck round-bottom flask equipped with a reflux condenser and a magnetic stirrer under anhydrous conditions. The reaction vessel was slowly heated to reflux and Compound 412 of FIG. 1C (6.10 mmol) dissolved in anhydrous THF (7 mL) was slowly added thereto with a syringe under nitrogen atmosphere. The resulting mixture was stirred at room temperature for about 1 hour to obtain a brown solution. The brown solution was transferred to a 100 mL two-neck round-bottom flask containing anhydrous Cu(I)Br (1.82 g, 12.7 mmol) with a metal tube. The mixture was stirred at room temperature for about 1 hour, and then dioxane (10 mL) was added thereto. The resultant was stirred at room temperature for about 1 hour to obtain a pale brown solution. The resulting compound (Compound 413 of FIG. 1C) was used in a subsequent reaction without purification due to its sensitivity to air and humidity.

### SYNTHESIS EXAMPLE 12

### Procedure 430 of FIG. 1C

2,4,6-Trichloro-1,3,5-triazine (150 mg, 0.68 mmol) dissolved in anhydrous toluene (25 mL) was added to the brown solution (Compound 413 of FIG. 1C) prepared in Synthesis Examples 11. The reaction mixture was stirred for about 6 days while maintaining it at about 100° C. After the reaction was completed, the product was filtered with a column chromatography tube containing an activated carbon (200-300 meshes) to remove solids. The filtrate was concentrated. The concentrated product was purified by a silica gel chromatography with a solvent system of 20% dichloromethane and hexane. As a result, white solid Compound IIe (1.05 g, yield: 49%), which was a 2,4,6-tris-perfluorophenylene-[1,3,5]triazine derivative, was obtained. The product was identified to have the same structure as that of Compound IIe through MS spectrum (MALDI-TOF).

MS (MALDI): m/e: 2613.9 (100.0%), 2612.9 (84.8%), 2614.9 (58.4%), 2615.9 (22.5%), 2616.9 (6.5%), 2617.9 (1.5%)

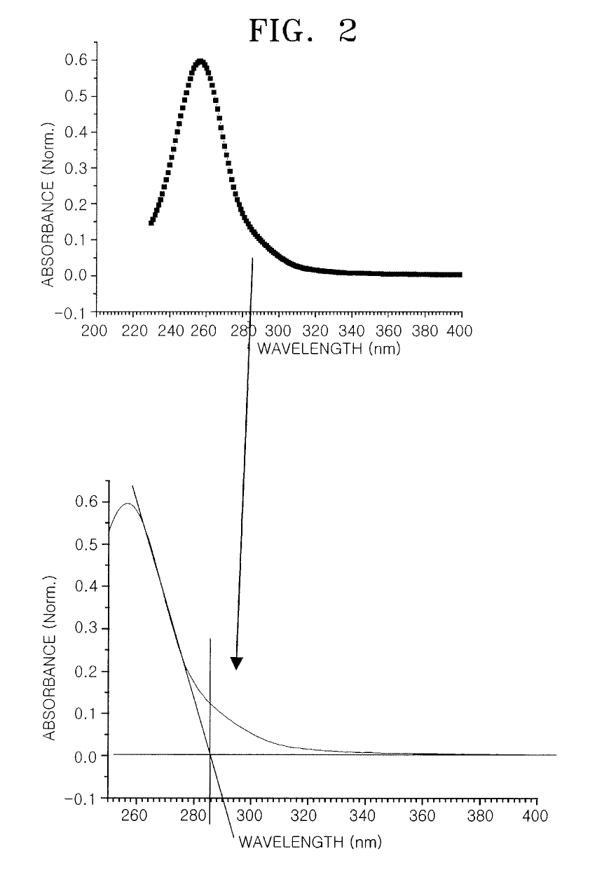

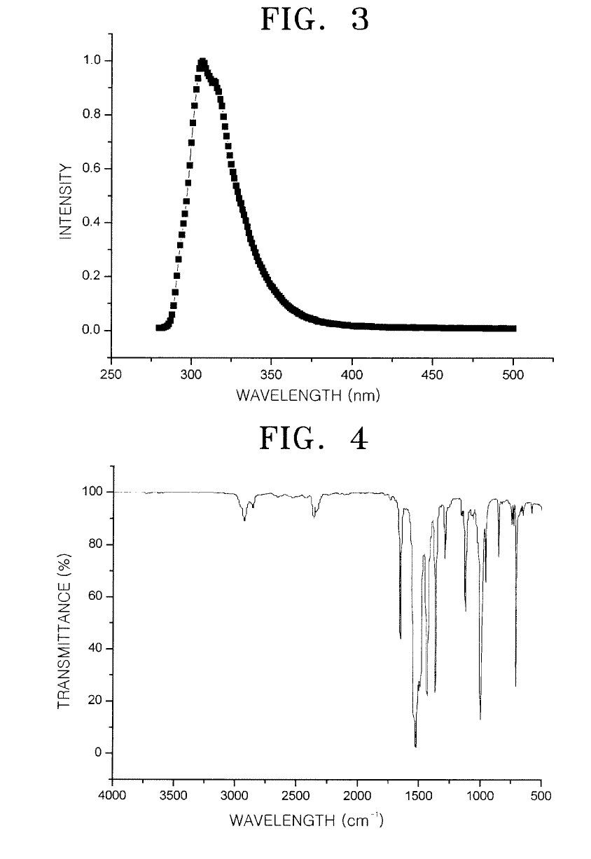

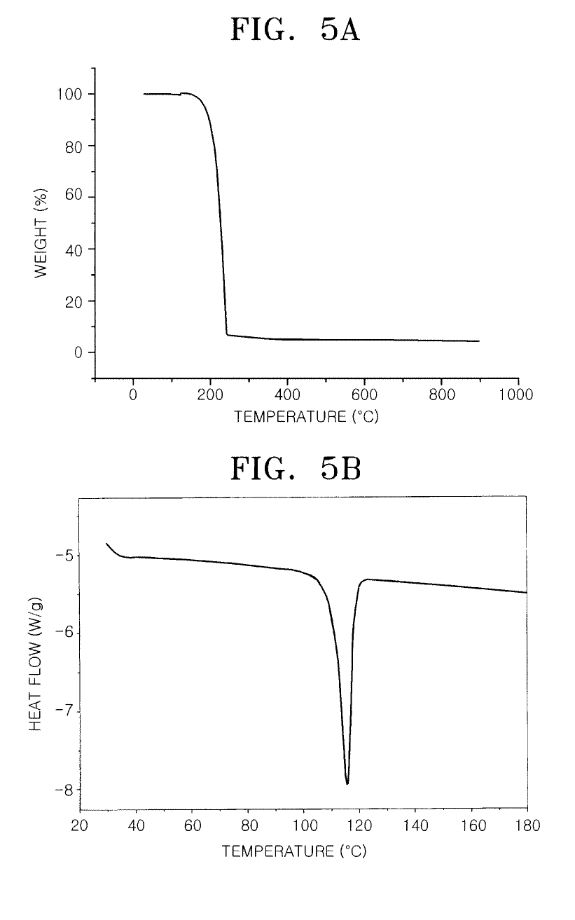

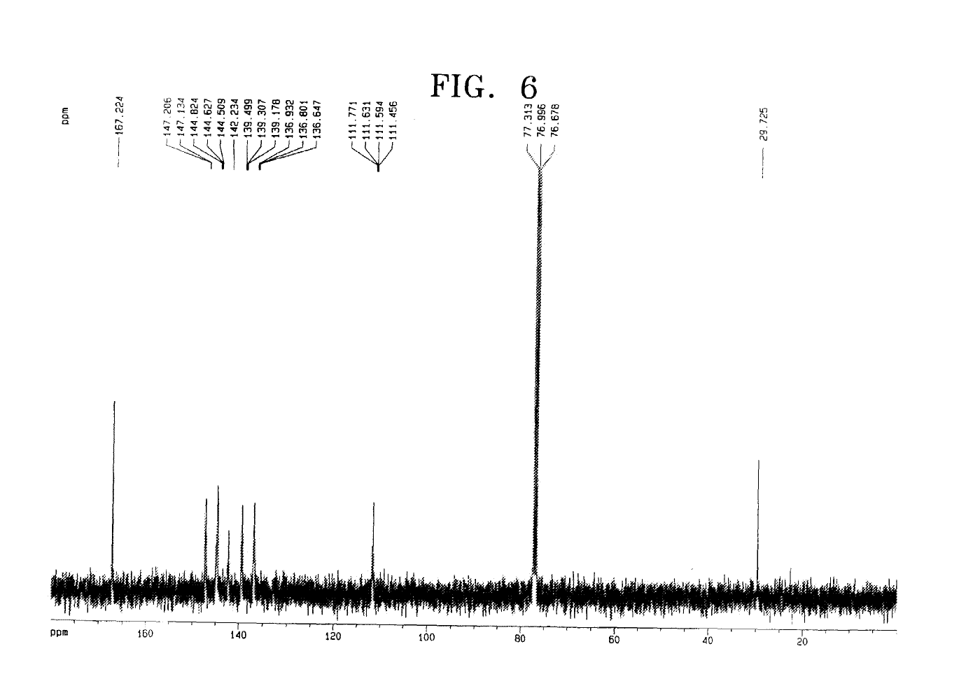

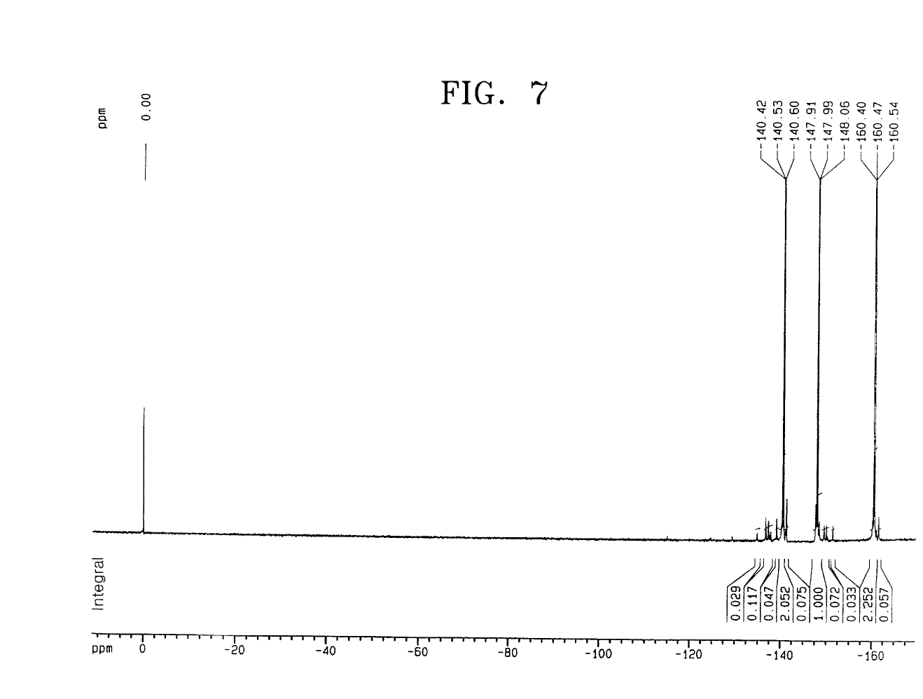

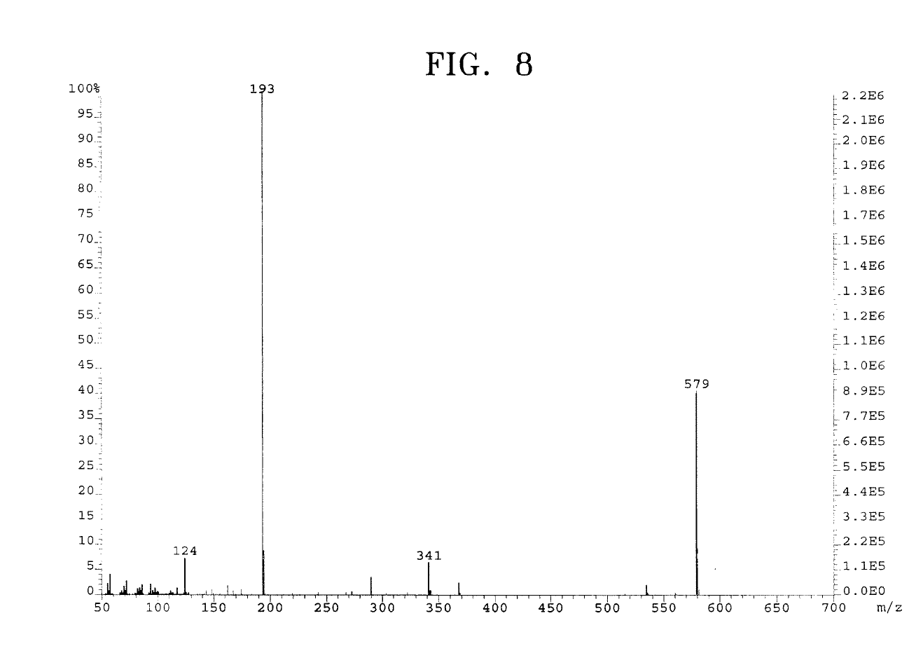

FIGS. 2 through 8 illustrates spectrums of Compound IIa synthesized in Synthesis Example 6, obtained by various analysis methods.

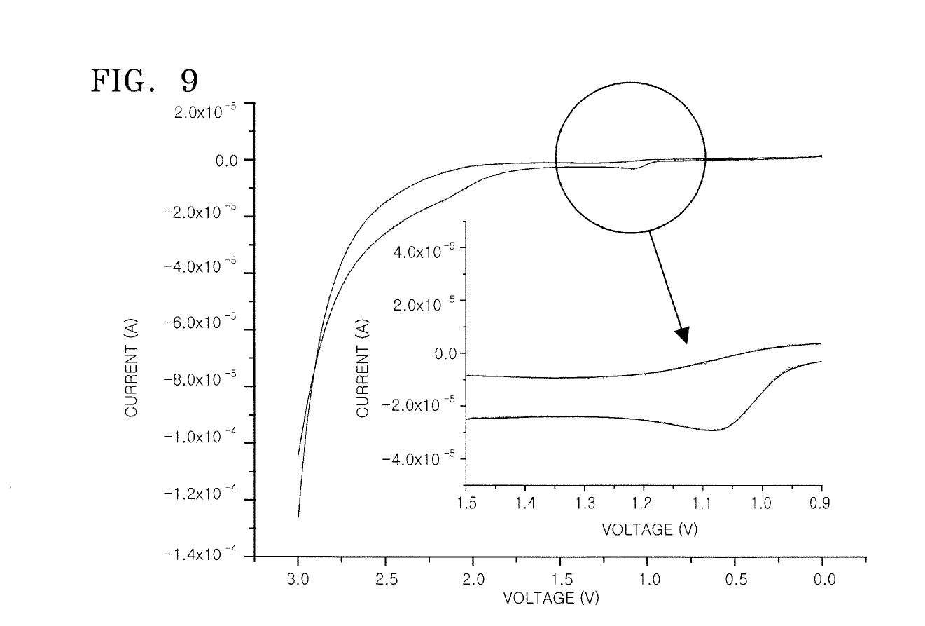

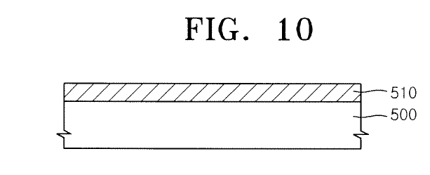

FIG. 9 illustrates cyclic voltametry (CV) results of Compound IIa. Of a highest occupied molecular orbital (HOMO) energy level and a lowest unoccupied molecular orbital (LUMO) energy level of Compound IIa, the HOMO value can be calculated using oxidation potential of CV data of FIG. 9. The correlation between oxidation potential (Ep: V) and energy level (eV) is established by comparing values known from a literature (Ichiro Imae, et al., Designed Monomers and Polymers, vol. 7, 127-133, 2004) or results previously measured by experiments. In the present invention, the HOMO value was obtained in the same manner as that described in reported literature. That is, the oxidation potential (Ep) of Compound IIa in FIG. 9 was 1.07 eV, and thus 5.32 eV was added thereto to obtain the HOMO value of 6.4 eV.

A HOMO-LUMO energy gap can be calculated using a band gap from onset of the UV spectrum of FIG. 2. The HOMO-LUMO energy gap is obtained from an equation, ΔE (HOMO-LUMO, eV)=hc ε/λ. In FIG. 2, λ is 290 nm, and thus 1240/290=4.28. Thus, the LUMO energy value at vacuum level is 2.12 eV. Generally, as the phenylene substituent lengthens, the LUMO value tends to increase. Using this tendency, the HOMO and LUMO values can be controlled. In the present invention, the HOMO level is 6.0 eV or more, and the LUMO level can be controlled within the range of 2-4 eV according to the length of the phenylene substituent. Thus, the compound according to an embodiment of the present invention can be appropriately applied to n-type organic semiconductor devices according to types and using purposes thereof.

As described above, the novel 2,4,6-tris-perfluorophenylene-[1,3,5]triazine compounds according to an embodiment of the present invention have lower energy levels (HOMO, LUMO) than any conventional n-type organic semiconductor material due to the triazine group having an electron-attracting property, and thus, can be applied to n-type organic semiconductor devices which require low energy levels.

Formation of an Organic Semiconductor Thin Film

FIG. 10 is a cross-sectional view for explaining a method of forming an organic semiconductor thin film according to an embodiment of the present invention.

Referring to FIG. 10, an organic semiconductor thin film 510 according to an embodiment of the present invention is formed on a substrate 500 by various thin film processing technologies. For application to various optical or electric devices, various thin film processing technologies, which can form the organic semiconductor thin film 510 composed of the novel compounds obtained in the above Synthesis Examples on the substrate 500, are required. The substrate 500 may be composed of, for example, ITO/glass, metal electrode/glass or metal electrode/silicon. The thickness of the thin film generally ranges from 1 nm to 1 μm. In the present invention, the organic semiconductor thin film 510 has preferably a thickness of about 10-500 nm. The organic semiconductor thin film 510 can be formed by various methods as described below.

(1) Vacuum Deposition

Of the organic semiconductor compounds according to an embodiment of the present invention, the compounds represented by Formula (1) to (4), wherein n is a number from 0 to 6, can be vacuum deposited on the substrate 500. The degree of vacuum in a vacuum chamber is remained at 10⁻⁷-10⁻⁸ Torr.

(2) Spin Coating

Of the organic semiconductor compounds according to an embodiment of the present invention, the compounds represented by Formula (1) to (4), wherein n is a number from 2 to 20, can be spin coated on the substrate 500. Examples of a solvent useful in this method include dichloromethane, chloroform, cyclohexanone, toluene, xylene, 1,1,2-trichloroethane, chlorobenzene, dichlorobenzene, nitrobenzene, dinitrobenzene and dimethylformaldehyde. In particular, polyphenylene, polyfluorene, polythiophene, polyphenylenevinylene, polyvinyl carbazole, polypyrrole, polyacetylene, polyaniline, and so on can be added for control of viscosity of an organic solution.

(3) Ink-Jet Coating

The organic semiconductor compounds according to an embodiment of the present invention can be ink-jet coated on the substrate 500 using the solvents listed above (2). The vapor pressure is maintained within a range of about 0.01-3.0 Kpa (0.1-22.5 mmHg) at 25° C.

(4) Screen Printing

The organic semiconductor compounds according to an embodiment of the present invention can be screen printed on the substrate 500 using the solvents listed above (2) to form an organic semiconductor thin film according to an embodiment of the present invention. When the organic solvent is used as a printing ink, the compound according to an embodiment of the present invention is added to the organic solvent in an amount of about 0.03-1.0 wt %. In an amount below 0.03 wt %, no effect is obtained. In an amount above 1.0 wt %, the effect of drying the printing ink is sufficient, but printability is deteriorated. The amount of the compound according to an embodiment of the present invention is most preferably about 0.03-0.5 wt %.

Although the methods of forming the organic semiconductor thin film according to an embodiment of the present invention have been described above, the present invention is not limited thereto. Those of ordinary skill in the art can recognize that various methods besides above-described methods can be applied in order to form the organic semiconductor thin film according to an embodiment of the present invention.

The present invention provides compounds for organic semiconductor devices, having a triazine group rather than a benzene group, which is commonly known, as a central molecular group. In the present invention, novel 2,4,6-tris-perfluorophenylene-[1,3,5]triazine compounds are prepared by coupling a fluorine-substituted phenyl group to a triazine compound, which has a π-electron attracting property, without difficulty in synthesis as in conventional technologies.

The compounds according to an embodiment of the present invention have lower energy levels (HOMO, LUMO) than any conventional n-type organic semiconductor material due to the triazine group having an electron-attracting property, and thus can be applied to n-type organic semiconductor devices in which require low energy levels. From the results of UV and CV measurements, the HOMO level is 6.0 eV or more, and the LUMO level ranges 2-3 eV at vacuum level. The LUMO value tends to increase as the substituent lengthens. Thus, the compound according to an embodiment of the present invention can be appropriately applied to n-type organic semiconductor devices according to types and using purposes thereof. The compounds for organic semiconductor devices can be used, for example, as electron injection/transport layers in an organic electroluminescent device, as an n-type channel material in an organic thin film transistor device, and as an n-type semiconductor material in a p-n type organic solar light device.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A compound for organic semiconductor devices represented by the following Formula: 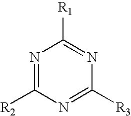
where each of R₁, R₂ and R₃ is a perfluorophenylene derivative represented by the following Formula: 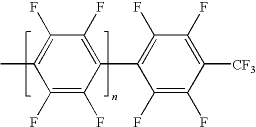
where n is an integer from 0 to 20.

2. An organic semiconductor thin film composed of the compound of claim 1.

3. An organic semiconductor device comprising a semiconductor film composed of the compound of claim 1.

4. The organic semiconductor of claim 3, wherein the semiconductor film constitutes an electron injection layer or electron transport layer of an organic electroluminescent device.

5. The organic semiconductor of claim 3, wherein the semiconductor film constitutes a channel layer of an n-type transistor.
